(19) United States
(12) Patent Application Publication
(10) Pub. No.: US 2010/0170323 A1
(43) Pub. Date: Jul. 8, 2010

(54) COMBINED DEVICE FOR ANALYTICAL MEASUREMENTS

(75) Inventor: John R.B. Lighton, Las Vegas, NV (US)

Correspondence Address: LEWIS AND ROCA LLP, 1663 Hwy 395, Suite 201, Minden, NV, 89423, US

(73) Assignee: SABLE SYSTEMS INTERNATIONAL, INC.

(21) Appl. No.: 12/727,372

(22) Filed: Mar. 19, 2010

Related U.S. Application Data
(62) Division of application No. 11/837,424, filed on Aug. 10, 2007.
(60) Provisional application No. 60/822,072, filed on Aug. 10, 2006.

(51) Int. Cl. G01N 33/497

(52) U.S. Cl. 73/23.3

(57) ABSTRACT

A method for measuring the concentration of a component gas in a gas stream in the presence of water vapor comprises introducing a gas stream into a gas analyzer, measuring in the gas analyzer the concentration of the component gas and storing a value representing the measured concentration, measuring the water vapor pressure in the gas analyzer and storing a value representing the measured water vapor pressure; and measuring the total pressure of the gas stream and storing a value representing the measured total pressure of the gas stream.

### CROSS-REFERENCE TO RELATED APPLICATIONS

This application is a divisional of co-pending U.S. patent application Ser. No. 11/837,424, filed Aug. 10, 2007, which claims the benefit of U.S. Provisional Patent Application Ser. No. 60/822,072, filed Aug. 10, 2006, the entirety of both are incorporated by reference herein.

### TECHNICAL FIELD

This invention relates to measurements of concentrations of oxygen, carbon dioxide, temperature and several other parameters with an apparatus hardened for field use.

### BACKGROUND

For field-portable work in aerial respirometry, several traditionally independent pieces of equipment were required. A pump provided motive force for air flow. A separate mass flow controller controlled STP-corrected air flow rate, which was subsampled by another pump and flow control system if required. An oxygen analyzer measured oxygen concentrations. A carbon dioxide analyzer measured carbon dioxide concentrations. A water vapor analyzer measured water vapor concentrations, and so forth. Additionally, a data acquisition system was required that communicated with a computer or retained raw data for later transfer to a computer. Often no final calculations of respiratory data could be performed in the field. Deploying and powering these multiple devices in the field was a nightmare, often obstructing the intended research or application. Merely jamming disparate instruments together in a so-called “metabolic cart” was not satisfactory for field portability and ruggedness. In addition, such units were generally oriented to clinical use on humans, and their operating assumptions are usually grossly simplistic and inflexible, making them unsuitable for publication-quality research. Ideally, a highly integrated, truly portable, self contained, versatile, highly portable and highly rugged instrument was needed.

Strangely, the need for a research grade, field-oriented, integrated respirometry instrument was not addressed by any firm. Eventually, a first attempt at such a system was introduced—the Sable Systems International FOXBOX© analytical instrument for measuring carbon dioxide and oxygen. But it still did not solve the problem of multiple instruments: one device for carbon dioxide and oxygen and an additional device for water vapor and other measurements. In addition, it had a limited flow capacity.

### SUMMARY OF THE INVENTION

The present invention solves the aforementioned problems and shortcomings of the existing art and solves other problems not listed above which will become apparent to those skilled in the art upon reading and understanding the present specification and claims.

In one embodiment, the present invention is a system for performing respirometry in the field. This system includes the following: a) a sample inlet; b) a filter connected to the sample inlet and to a mass flow meter, to prevent the entry of particulate matter; c) a pump connected to the mass flow meter; d) a plurality of analyzers connected to the pump to maintain flow through the analyzers, the plurality of analyzers having at least i) an oxygen analyzer; ii) a carbon dioxide analyzer; iii) a barometric pressure measurement means, plumbed either to ambient pressure, pressure within the flow system, or to both; iv) a water vapor analyzer to measure relative humidity (RH) in the sampled air; and v) a temperature sensor near the water vapor analyzer; e) a data acquisition system that translates into digital form data from the plurality of analyzers; f) a data management system that incorporates data from the analyzers and performs calculations to at least determine percentages of oxygen and carbon dioxide corrected for water vapor pressure; g) a control system integrated into the apparatus to provide user control over at least some of the calculations; and h) a display for readouts of the plurality of analyzers and results of the calculations.

In another embodiment, the system's oxygen analyzer is a fuel cell, or a zirconia cell. Alternately, the system's oxygen analyzer is based on a measurement principle, such as paramagnetism or optical fluorescence. Additionally, the system can provide calculation of the saturated water vapor pressure obtained at the site of the RH analyzer. As another option, the system can provide conversion of the corrected or uncorrected RH to water vapor activity and then multiply by saturated water vapor pressure to determine the actual water vapor pressure obtained at the RH sensor. As another option, the system can combine the barometric pressure with the water vapor pressure to yield a proportionality coefficient which, when multiplied by the concentration of any measured gas species, compensates for the dilution effect of water vapor. As another option, the system can correct dilution-corrected gas concentrations to STP using the barometric pressure data. Additionally, the system can convert primary volumetric flow rate to standard temperature and pressure using the barometric pressure and the temperature. Optionally, the system can provide automated or manual methods of measuring and storing oxygen and carbon dioxide baseline values. Alternately, the pump can be connected between the filter and the mass flow meter.

In another embodiment, an apparatus can include the following: a) a sample inlet; b) a filter connected to the sample inlet and to a mass flow meter, to prevent the entry of particulate matter; c) a pump connected to the mass flow meter; d) a plurality of analyzers connected to the pump to maintain flow through the analyzers, the plurality of analyzers comprising at least i) an oxygen analyzer, based on any measurement principle, such as fuel cell, paramagnetic, zirconia cell, or optical fluorescence; ii) a carbon dioxide analyzer; iii) a barometric pressure measurement means, plumbed either to ambient pressure, pressure within the flow system, or to both; iv) a water vapor analyzer to measure relative humidity in the sampled air; and v) a temperature sensor near the water vapor analyzer; e) a data acquisition system that translates into digital form data from the plurality of analyzers; f) a data management system that incorporates data from the analyzers and performs calculations; g) a control system integrated into the apparatus to provide user control over at least some of the calculations; h) a means integrated into the apparatus to display readouts of the plurality of analyzers and results of the calculations; i) a means for calculating the saturated water vapor pressure obtained at the site of the RH sensor; j) a means for converting the corrected or uncorrected RH to water vapor activity and then multiplying by saturated water vapor pressure to determine the actual water vapor pressure obtained at the RH sensor; k) a means for combining the barometric pressure with the water vapor pressure to yield a proportionality coefficient which, when multiplied by the concentration of any measured gas species, compensates for the dilution effect of water vapor; l) a means for correcting dilution-corrected gas concentrations to STP using the barometric pressure data; m) a means for converting primary volumetric flow rate to standard temperature and pressure using the barometric pressure and the temperature; and n) a means for use in either automated or manual methods of measuring and storing oxygen and carbon dioxide baseline values.

Further details and advantages of the present invention will be appreciated by reference to the figures and description of exemplary embodiments set forth herein.

### BRIEF DESCRIPTION OF THE DRAWINGS

The above noted and other features of the invention will be better understood from the following detailed description when considered with reference to the accompanying drawings.

FIG. 1 is an overview diagram of the apparatus.

FIG. 2 is a diagram of the high flow rate subsystem of FIG. 1.

FIG. 3 is a diagram illustrating the central processing unit of the apparatus, showing signal and interface inputs (right-facing arrows) and outputs (left-facing arrows).

FIG. 4 is a flow diagram showing the principal steps performed by the apparatus.

### DETAILED DESCRIPTION

In the following detailed description, references made to the accompanying drawings which form a part hereof and in which is shown by way of illustration specific embodiments in which the invention may be practiced. These embodiments are described in sufficient detail to enable those skilled in the art to practice and use the invention, and it is to be understood that other embodiments may be utilized in that electrical, logical, and structural changes may be made without departing from the spirit and scope of the present invention. The following description is, therefore, not to be taken in a limiting sense and the scope of the present invention is defined by the appended claims and equivalents thereof.

The inventive field apparatus satisfies not only the need for oxygen and carbon dioxide measurement in a single protected apparatus, dustproof and waterproof for travel, but also the need to further incorporate a water vapor analyzer, for immediate correction of water vapor dilution. The first advantage is fewer devices to transport and set up. Because interconnections are internal and optimized, reliability is a greatly enhanced second improvement. Furthermore, real-time barometric pressure measurement not only corrects the gas analyzer readings to standard temperature and pressure, but also corrects for the dilution effect of water vapor and such is automatically incorporated into the 02 or CO2 analysis, which increases the accuracy and reproducibility of the data, the third and fourth improvements. If the researcher could not compensate the data immediately in the field, the researcher frequently discovers the need for another visit to the field, a fifth improvement. Thus, on-site data compensation can be a major savings in researcher time and travel costs. In addition, real-time correction for water vapor dilution eliminates the need for expensive, toxic and short-lived scrubber chemicals, which are additional improvements.

In addition, the respirometer has an option of either a pneumotachometer, a respirometer mask or an enclosed chamber, depending on the way the operator configures the instrument. Further, the apparatus is designed for greater mass flow and control for mask or chamber applications. The high-flow capacity permits the testing of large organisms, such as elephants, sea lions and humans. The apparatus also incorporates internal data logging with real-time calculation of V02, VCO2, and respiratory quotient (RQ), without the need for downloading to a separate computer for calculations. Temperature measurements can be external and internal. In one embodiment there is porting for internal temperature regulation. The apparatus also can accept voltage and resistance analog inputs which can, for example, be used to input pneumotachometer flow rates and organism temperatures.

The new apparatus is an integrated research grade instrument that includes a precision miniature pump, a mass flow meter, an oxygen analyzer and carbon dioxide analyzer. The mass flow meter could have a range of 20-2000 ml/min. In addition, the apparatus, in a preferred embodiment, includes a high flow rate mass flow system (range 5-100 or 20-500 liters/minute). The apparatus preferably is accurate, stable and has a high resolution data acquisition system. Stability is shown by the short-term oxygen noise of typically less than 0.001% root mean square (RMS) and the long-term oxygen drift is typically 0.002% RMS at constant temperature. Short-term carbon dioxide noise is typically less than 0.0005% (5 ppm) RMS; the long-term carbon dioxide drift is typically less than 0.002% RMS near zero concentration at constant temperature.

The apparatus can be the nucleus of a research-grade field respirometry system, but is also suitable in the conventional research or teaching laboratory or any allied purpose. The apparatus can be provided with the stability and flow rate range to handle large creatures, such as elephants, sea lions and humans. It is designed to accommodate push-mode respirometry, pull-mode respirometry, general oxygen or carbon dioxide sampling in air or gas streams of any type, and constant volume respirometry. It also can be used in fuel/air analysis, geological prospecting and many other applications. For users in many fields, it provides accurate oxygen and/or CO2 measurement combined with toughness and portability.

In one embodiment, the apparatus also has the following additional features. It is accurate up to 0.1% over the range of 1-100% oxygen. The display shows oxygen to 0.001% (one part in 100,000). In one embodiment, carbon dioxide can be displayed to 1 ppm or 0.001% depending on the range selected. Carbon dioxide can be measured in multiple ranges from 100 ppm to 10%. Oxygen calibration only requires atmospheric air. In one configuration, the apparatus is fully temperature, water vapor and barometric pressure compensated. The apparatus is microcomputer-based, intelligent and auto-diagnosing. The apparatus displays barometric pressure to 1 Pa (or one part in 100,000). The apparatus can have a four-line alphanumeric display with selectable backlighting or other conventional display.

Preferably the apparatus is designed to conserve energy and will operate about 10 hr on one battery charge. The apparatus can also be powered from a DC supply of 12-17 V. In another embodiment, the apparatus can be operated with photovoltaic power or from alternating current with a suitable adapter.

In preferred embodiments, the apparatus is fully portable. Preferably the apparatus is 12V DC powered from a battery that is rechargeable by the user. The apparatus optionally offers multiple analog (for example, 0-5V) outputs and a digital (RS-232) output. In one embodiment, the apparatus measures a fuel/air ratio.

In another embodiment, the apparatus has serial output capability which requires no specialized voltage measurement interfaces. Its serial output is preferably in ordinary ASCII characters that can be received by any terminal program on any PC or Mac computer. Preferably the apparatus transmits up to ten serial strings per second from its serial port, the interval of which is optionally controlled by the user. In this form the data can be loaded or pasted into a variety of statistical packages, spreadsheets or specialized analysis programs. In one embodiment, the apparatus stores up to 8000 data points, each of which contains all variables measured thereon, also at intervals set by the user. Data are preferably uploaded via a high speed data link in seconds. In another embodiment there are four analog outputs to use with a voltage-input data acquisition system. Preferably these analog outputs have high resolution (16 bits), and users can switch between various ranges.

In another embodiment, two accessory inputs can operate in voltage-measuring mode for recording external instruments in the field. In yet another embodiment, the input can be operated in temperature mode with thermistor temperature probes, available in 0.2 or 0.1° C. accuracy. Another input option is the resistance-measuring mode for any resistive sensor, such as a light dependent resistor to monitor animal movement or activity. Another input option is the voltage-measuring mode (range 0-5V at 16 bits resolution).

Preferably calibration of the apparatus is straightforward, using a span gas of known oxygen concentration, such as dry ambient air that typically has an oxygen concentration of 20.9%. In one embodiment, no zero-adjustment is necessary because internal electronics continuously adjust its zero point using ambient air. Optionally the user can manually zero-adjust the apparatus. Like all such analyzers, the carbon dioxide can be calibrated with CO2-scrubbed air or nitrogen and a CO2 span gas.

In a preferred embodiment, there is a method of generating an STP-corrected mass air flow rate. Other analyzers run a pump at a high speed then use a conventional mass flow controller to bleed off the appropriate mass flow rate against the high pressure head. With this invention, high speed computational circuitry continuously measures the mass flow rate of the air (adjusted for STP and water vapor) passing through the pump and adjusts the speed of the pump to deliver the precise flow rate requested by the user. Flow rate is set with driftless digital controls backed by nonvolatile memory. The apparatus affords a sample flow rate over a wide range of 20 to 2000 ml/minute, and in a preferred embodiment, a primary mass flow range of 5-100 or 20-500 liters/minute. This technique reduces power consumption and increases pump diaphragm life. Flow rate variance is typically 2% of the reading. This performance is better than the conventional “percent of full scale” and is equivalent to that of a separate pump coupled to a premium mass flow control valve.

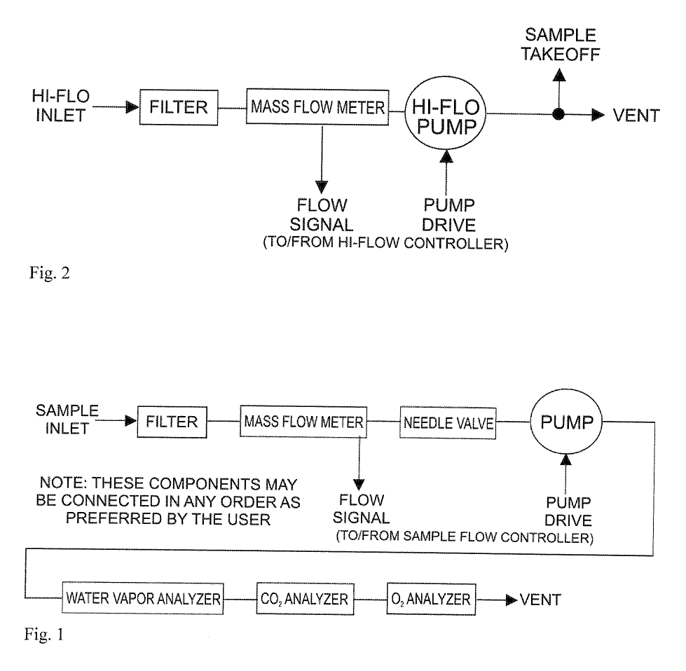

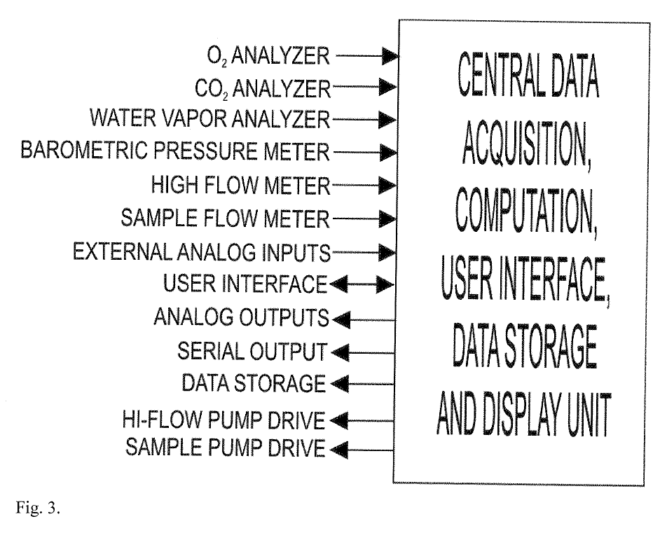

FIG. 1 is an overview of the apparatus 2. At one end of the apparatus 2 is a sample inlet 10, which connects to a filter 20, which filters out particles to eliminate fouling of the analyzers. In this embodiment, the filter 20 is connected to a mass flow meter 30. The mass flow meter 30 produces a signal 40 which is conveyed to the sample flow controller (not shown). The gas passes through the mass flow meter 30 through a connection such as a needle valve 50 to a pump 60, which is operated by a pump drive 70. The pump 60 moves the test gas to the analyzers. In this embodiment, the gas first enters the water vapor analyzer 80, and then a carbon dioxide analyzer 90, and finally an oxygen analyzer 100 before the test gas leaves the apparatus 2 through a vent 200. The water vapor analyzer 80 measures the percent of relative humidity (RH %) in the sampled air stream. A temperature sensor (see below) is located in close proximity to the relative humidity sensor of the water vapor analyzer 80. The input and output plumbing connections of all the components shown in this figure are brought out to the front panel, allowing the user to connect the various components in any order as preferences and/or requirements dictate. The order of the components shown in FIG. 1 is one preferred configuration. In this configuration, the sample inlet 10 is connected directly to the high-flow sample take-off (FIG. 2) for standard mask or chamber respirometry, or to a mask with a pneumotachometer, with the pneumotachometer voltage output being connected to an analog input (FIG. 3); or to a mask or chamber in the case of a small animal, the metabolic rate of which can be measured at a mass flow rate of two liters/minute or less.

FIG. 2 shows the high flow rate subsystem of the apparatus. The mass flow meter 30 may be placed before or after the pump 60. The high flow control unit 110 can be an autonomous controller with its own setpoint and other controls (preferred configuration), or it may be integrated into the central data processing unit (discussed below). After the pump 60, there is an optional valve 120 to direct the test gas to a sample takeoff 130, or to the vent 200.

FIG. 3 is a schematic of the central processing unit 210 of the apparatus 2, showing signal and interface inputs (left-facing arrows) and outputs (right-facing arrows). Inputs come from the oxygen analyzer 100, the carbon dioxide analyzer 90, the water vapor analyzer 80, a barometric pressure meter 220, the high flow meter 30, the sample flow meter 230, and a plurality of various optional other external analog inputs 240. Outputs from the CPU 210 are directed to a user interface 250, various analog output(s) 260, serial output(s) 270, external data storage 280, a high-flow pump drive 70, a sample pump drive 290 and a display unit 300.

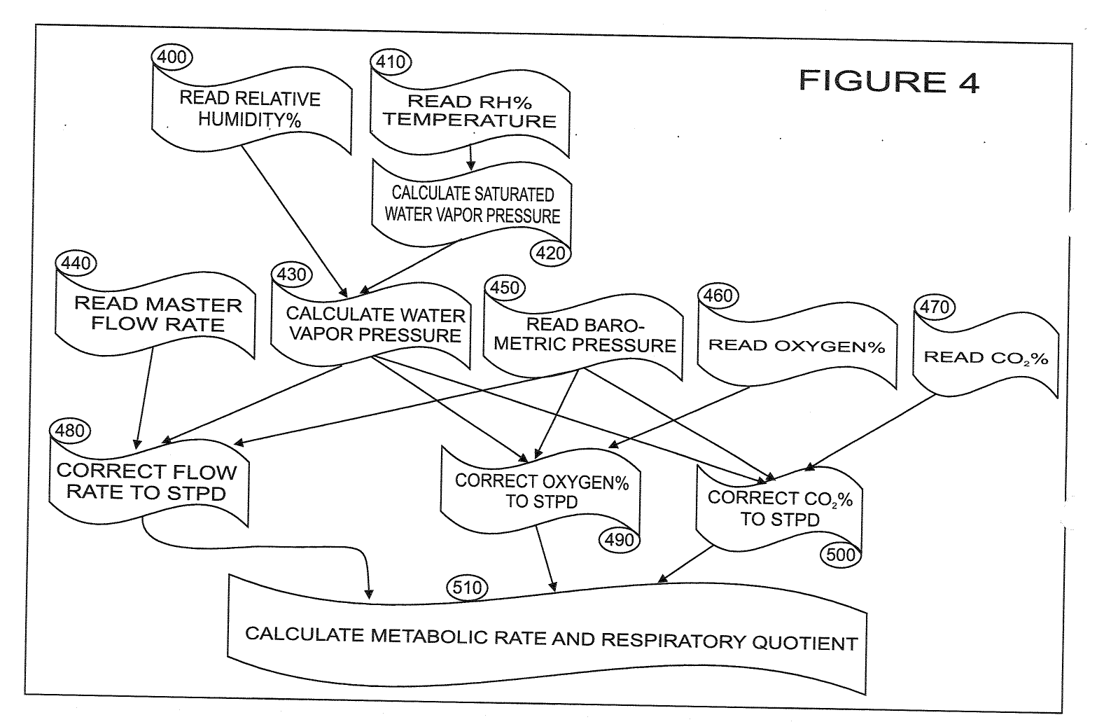

FIG. 4 is a flow diagram showing the principal steps taken by the apparatus to measure metabolic rate and respiratory quotient without requiring expendable chemicals. FIG. 4 illustrates the steps performed by the CPU 210 in accordance with a program, or in the alternative, a hardware circuit designed to make the same iterative calculations. First, the readings of relative humidity % (RH %) 400 and temperature 410 are used to calculate the saturated water vapor pressure 420, which are used in turn to calculate water vapor pressure 430. The water vapor pressure calculation 430, the master flow rate reading 440, and the barometric pressure reading 450 are used to correct flow rate to STPD 480. 02% reading 460 and CO2% reading 470 are combined with the calculated water vapor pressure 430 and the barometric pressure reading to correct the 02% to STPD 490 and the CO2% to STPD 500. Lastly, in the current embodiment, the corrected flow rate 480, corrected 02% 490 and corrected CO2% are used to calculate the metabolic rate and respiratory quotient 510.

There are many variations contemplated for this field apparatus for determine metabolic rate and respiratory quotient. First, there can be a means of producing or measuring a primary air flow into which is expired air from a subject organism, whereby the organism's own inspiration and expiration or by means of a forced air flow extrinsic to the organism; optionally this air flow can be coupled to a means of controlling the mass or volumetric flow rate. Optionally, the means of determining the mass or volumetric air flow via a mass or volumetric flow measurement device includes, but is not limited to, a mass flow meter or pneumotachograph. There also can be a sample takeoff 130 to direct a sub-sample of the sample gas or the larger flow rate through the gas analyzers 80, 90, 100, etc.

The barometric pressure measurement meter 220 can be plumbed either to ambient pressure, pressure within the flow system, or both. The measured temperature at the RH sensor can be used to correct the measured RH to cancel any known temperature dependency of RH on temperature at 410 or 420 of FIG. 4. The measured temperature of the RH sensor 80 may be used to calculate the saturated water vapor pressure at the site of the RH sensor 80. Also provided is a means to convert the corrected or uncorrected RH to water vapor activity, which is then multiplied by saturated water vapor pressure, thereby calculating the actual water vapor pressure obtained at the RH sensor 80. Also provided is a means to combine the barometric pressure 450 with the water vapor pressure 430 to yield a proportionality coefficient which, which multiplied by the oxygen concentration 460 or the carbon dioxide concentration 470 compensates for the dilution effect of water vapor to produce water-vapor-corrected 02% and CO2%, respectively. Means also can be provided to convert these two corrected gas concentrations to STP using the barometric pressure data from meter 220. Means also can be provided to convert the primary flow rate reading 440, if volumetric in nature, to the standard temperature and pressure (or mass flow) equivalent using the barometric pressure 450 and the air stream temperature.

The data acquisition portion of the CPU 210 translates into digital form all input, including all input from high flow meter 30, water vapor analyzer 80, carbon dioxide analyzer 90, oxygen analyzer 100, barometric pressure meter 220, sample flow meter 230 and other external analog inputs 240. The CPU 210 may include a data management system that incorporates the acquired data and performs at least the calculations described above. Optional calculations include but are not limited to calculations of a) oxygen consumption by an organism, b) carbon dioxide production rate by an organism, c) of respiratory quotient of an organism, and d) actual heat production or true aerobic metabolic rate.

Robust standardization capabilities are optional in the apparatus. For oxygen and carbon dioxide, there can be either or both automated and manual methodologies to measure and store for calculation the baselines of the primary or samples air flows. Optionally, there can also be either or both automated and manual methodologies to measure and store for calculation the spans of the oxygen and carbon dioxide analyzers. Optionally, there can be either or both automated and manual methodologies to measure and store for calculation the carbon dioxide zero point of the carbon dioxide analyzer. Such methodologies for measuring and storing data for baselining, spanning and zeroing can be provided with other inputs, including but not limited to the water vapor analyzer 80, the high flow meter 30, the barometric pressure meter 220, and the sample flow meter 230.

Of course, the apparatus 2 incorporates an operator control system, or user interface 250, optionally integrated or running on an external computer, to provide full operator control over the operation of the apparatus 2, including optionally all of the abovementioned calculations, baselining, and (if applicable) spanning and zeroing using stored results of the various inputs.

In addition, the apparatus 2 features a display unit 300, optionally integrated or running on an external computer, by which the results generated by the apparatus may be displayed. Any standard display can be used therein.

Although specific embodiments have been illustrated and described herein, it will be appreciated by those of ordinary skill in the art that any arrangement which is calculated to achieve the same purpose may be substituted for the specific embodiment shown. For example, the display described above, need not be integral to the apparatus, but can be a standard display of any type which the user attaches to the apparatus. This application is intended to cover any adaptations or variations of the specific invention. Therefore, it is manifestly intended that this invention be limited only by the claims and equivalents thereof.

What is claimed is:

1. A method for measuring the concentration of a component gas in a gas stream in the presence of water vapor comprising:
introducing a gas stream into a gas analyzer;
measuring in the gas analyzer the concentration of the component gas and storing a value representing the measured concentration;
measuring the water vapor pressure in the gas analyzer and storing a value representing the measured water vapor pressure; and
measuring the total pressure of the gas stream and storing a value representing the measured total pressure of the gas stream.

2. The method of claim 1 further including applying a correction factor to the value representing the measured concentration to compensate for the diluting effect of water vapor.

3. The method of claim 2 wherein applying a correction factor to the value representing the measured concentration to compensate for the diluting effect of water vapor comprises:
calculating a coefficient comprising the value representing the measured total pressure of the gas stream divided by the value representing the measured total pressure of the gas stream minus the value representing the measured water vapor pressure; and
multiplying the value representing the measured concentration by the coefficient.

4. The method of claim 1 wherein measuring in the gas analyzer the concentration of the component gas comprises measuring in the gas analyzer the concentration of oxygen.

5. The method of claim 1 wherein measuring in the gas analyzer the concentration of the component gas comprises measuring in the gas analyzer the concentration of carbon dioxide.

6. The method of claim 1 wherein measuring in the gas analyzer the concentration of the component gas comprises measuring in the gas analyzer the concentration of methane.

7. The method of claim 1 wherein the airstream comprises expired air from a subject organism.

8. A method for measuring the concentration of a component gas in a gas stream in the presence of water vapor comprising:
introducing a gas stream into a gas analyzer;
measuring in the gas analyzer the concentration of a first component gas and storing a value representing the measured concentration of the first component gas;
measuring in the gas analyzer the concentration of a second component gas and storing a value representing the measured concentration of the second component gas;
measuring the water vapor pressure in the gas analyzer and storing a value representing the measured water vapor pressure; and
measuring the total pressure of the gas stream and storing a value representing the measured total pressure of the gas stream.

9. The method of claim 8 further including;
applying a correction factor to the value representing the measured concentration of the first component gas to compensate for the diluting effect of water vapor; and
applying a correction factor to the value representing the measured concentration of the second component gas to compensate for the diluting effect of water vapor.

10. The method of claim 9 wherein:
applying a correction factor to the value representing the measured concentration of the first component gas comprise calculating a coefficient comprising the value representing the measured total pressure of the gas stream divided by the value representing the measured total pressure of the gas stream minus the value representing the measured water vapor pressure and multiplying the value representing the measured concentration of the first component gas by the coefficient; and
applying a correction factor to the value representing the measured concentration of the second component gas comprise calculating a coefficient comprising the value representing the measured total pressure of the gas stream divided by the value representing the measured total pressure of the gas stream minus the value representing the measured water vapor pressure and multiplying the value representing the measured concentration of the second component gas by the coefficient.

11. The method of claim 8 wherein:
measuring in the gas analyzer the concentration of the first component gas comprises measuring in the gas analyzer the concentration of oxygen; and
measuring in the gas analyzer the concentration of the second component gas comprises measuring in the gas analyzer the concentration of carbon dioxide.

12. The method of claim 8 wherein the airstream comprises expired air from a subject organism.
